(19) United States
(12) Patent Application Publication
(10) Pub. No.: US 2024/0066165 A1
(43) Pub. Date: Feb. 29, 2024

(54) HYDROGEN PEROXIDE VAPOR VAPORIZER

(71) Applicant: WOOJUNG BIO, INC., Suwon-si, Gyeonggi-do (KR)

(72) Inventors: Byung Nyun CHUN, Suwon-si, Gyeonggi-do (KR); Joo Hyung CHUN, Suwon-si, Gyeonggi-do (KR); Yun Jae JUNG, Osan-si, Gyeonggi-do (KR)

(21) Appl. No.: 18/270,050

(22) PCT Filed: Dec. 21, 2021
(86) PCT No.: PCT/KR2021/019465

§ 371 Date: Jun. 28, 2023

(30) Foreign Application Priority Data
Dec. 31, 2020 (KR) 10-2020-0189322

(51) Int. Cl. A61L 2/20

(52) U.S. Cl. CPC A61L 2/208; A61L 2202/10; A61L 2202/15

(57) ABSTRACT

A hydrogen peroxide vapor vaporizer, which is used in a sterilizer that sterilizes by using hydrogen peroxide vapor generated by vaporizing a hydrogen peroxide solution, comprises: a body part formed in the shape of a hollow column; a vaporization part which is provided at one side of the inside of the body part and has a predetermined capacity to accommodate a predetermined hydrogen peroxide solution; support parts for supporting the vaporization part to provide same to be spaced a predetermined distance apart from the inside of the body part; a heating part which is provided at one side of the vaporization part to heat the vaporization part; and a discharge part which is provided at the upper portion of the body part to induce discharging of the hydrogen peroxide vapor vaporized in the vaporization part.

### TECHNICAL FIELD

The present invention relates to a hydrogen peroxide vaporizer, more specifically to a hydrogen peroxide vaporizer that is capable of being simple in configuration and optimizing the vaporization efficiency of a hydrogen peroxide solution, thereby enabling efficient sterilization with hydrogen peroxide vapor.

### BACKGROUND ART

A chemical sterilizer is a device for performing a sterilization process using sterilants such as hydrogen peroxide gas, ethylene oxide gas, chlorine dioxide gas, and the like.

In a conventional chemical sterilizer using hydrogen peroxide as a sterilant, the hydrogen peroxide is vaporized in a sterilization chamber at a low pressure less than or equal to 3 torr. The vaporized sterilant is supplied to the interior of the chamber having a given volume to perform sterilization, and in this case, an amount of sterilant supplied is diffused at a pressure greater than or equal to 20 torr so that the sterilant is sufficiently supplied, while being kept in a state of gas at a predetermined temperature. Like this, only when the sterilant is kept to the state of gas, it is transferred to a sterilization article located in the interior of the chamber and the inside of the sterilization article, thereby performing successful sterilization.

A conventional plasma sterilizer generally has several liters of a sterilization chamber, and an amount of sterilant (hydrogen peroxide solution) supplied at a temperature of about 60° C. is several milliliters. The larger the capacity of sterilization chamber is, the larger the amount of hydrogen peroxide supplied is, so that an amount of sterilant to be vaporized in a vaporizer becomes increased. Therefore, there is a need to develop a vaporizer for allowing a hydrogen peroxide solution to have sufficient residence time therein and effectively heating and vaporizing the hydrogen peroxide solution.

### DISCLOSURE OF THE INVENTION

### Technical Problem

Accordingly, the present invention has been made in view of the above-mentioned problems occurring in the related art, and it is an object of the present invention to provide a hydrogen peroxide vaporizer that is capable of being simple in configuration and optimizing the vaporization efficiency of a hydrogen peroxide solution, thereby enabling efficient sterilization with hydrogen peroxide vapor.

The technical problems of the present invention are not limited as mentioned above, and other technical problems not mentioned herein will be obviously understood by one of ordinary skill in the art through the following description.

### Technical Solution

To accomplish the above-mentioned objects, according to the present invention, there is provided a hydrogen peroxide vaporizer for use in a sterilizer for performing sterilization with hydrogen peroxide vapor generated by means of the vaporization of a hydrogen peroxide solution, the hydrogen peroxide vaporizer including: a body part having the shape of a hollow column; a vaporization part disposed on one side of the inside of the body part and having a predetermined capacity to accommodate the hydrogen peroxide solution; support parts for supporting the vaporization part to allow the vaporization part to be spaced apart from the inside of the body part by a predetermined distance; a heating part disposed on one side of the vaporization part to heat the vaporization part; and a discharge part disposed on top of the body part to induce the hydrogen peroxide vapor generated from the vaporization part to be discharged.

In this case, the vaporization part may have protrusions or partition walls protruding from the inner bottom thereof or at least one or more wall surfaces thereof to increase the contact area with the hydrogen peroxide solution.

Further, the discharge part may become gradually narrow to the top from the bottom thereof.

The hydrogen peroxide vaporizer may further include a discharge fan disposed on one side of the inside of the body part to easily discharge the hydrogen peroxide vapor generated from the vaporization part to the discharge part.

Further, the discharge fan may be disposed at a relatively lower position than the vaporization part.

The hydrogen peroxide vaporizer may further include a solution supply part disposed on one side of the body part to supply the hydrogen peroxide solution to the vaporization part.

The hydrogen peroxide vaporizer may further include a diffusion fan disposed on top of the discharge part or spaced apart from top of the discharge part by a predetermined distance.

### Advantageous Effects of the Invention

Some advantages of the hydrogen peroxide vaporizer according to the present invention are as follows.

Firstly, the hydrogen peroxide vaporizer is simple in configuration and optimizes the vaporization efficiency of the hydrogen peroxide solution supplied to the vaporization part through the configuration of the vaporization part.

Secondly, the vaporization part has the wall surfaces with given height so that the hydrogen peroxide solution supplied to the vaporization part scatters from the bottom of the vaporization part and comes into contact with the wall surfaces of the vaporization part, thereby improving the vaporization efficiency of the hydrogen peroxide solution.

Thirdly, through the protrusions or partition walls of the vaporization part, the contact area of the hydrogen peroxide solution with the protrusions or partition walls becomes increased, so that the volume of the hydrogen peroxide vaporizer as well as the volume of the vaporization part is reduced, while the vaporization efficiency of the hydrogen peroxide solution is being sufficiently improved.

Fourthly, the area of the vaporization part is reduced, thereby enabling the whole volume of the hydrogen peroxide vaporizer and the sterilizer to be decreased.

Fifthly, through the optimized configuration of the discharge part, the hydrogen peroxide vapor is prevented from flowing back downward from the discharge part and minimized in a resistance to air flow, and further, the capacity rates of the discharge fan and the diffusion fan to the discharge part are optimized to maximize the diffusion efficiency of the hydrogen peroxide vapor.

Sixthly, the vaporization of the hydrogen peroxide solution and the diffusion of the hydrogen peroxide vapor are maximized, thereby optimizing the sterilization efficiency.

Seventhly, the vaporization part is fixed to the body part with a minimum contact point with the body part through the support parts, thereby enabling the heat loss generated when the heat supplied from the heating part comes into contact with external air by the body part to be minimized. Further, the vaporization part is spaced apart from the body part through the support parts, so that even if a fire happens in the vaporization part, the vaporization part, which is distant from surrounding combustible materials, ensures the safety of use.

Eighthly, the hydrogen peroxide vapor is mixed with high temperature vapor supplied from the discharge fan to prevent the hydrogen peroxide vapor from being cold and liquefied again, while moving, so that the hydrogen peroxide vapor is stably diffused up to a target.

Lastly, the hydrogen peroxide vaporizer is made of lightweight aluminum with high chemical resistance, thereby reducing the weight of the sterilizer as well as the weight thereof to the maximum.

The effectiveness of the present invention is not limited as mentioned above, and it should be understood to those skilled in the art that the effectiveness of the invention may include another effectiveness as not mentioned above from the detailed description of the present invention.

### BRIEF DESCRIPTION OF DRAWINGS

The following drawings herein illustrate preferred embodiments of the present invention and serve to facilitate the general understanding of the scope of the present invention, together with the detailed description of the present invention. Therefore, the present invention is not limitedly interpreted only with the attached drawings.

FIG. 1 is a block diagram showing a schematic configuration of a hydrogen peroxide vaporizer according to the present invention;

FIG. 2 is a side view showing a sterilizer having the hydrogen peroxide vaporizer according to the present invention;

FIG. 3 is a transverse sectional view showing a body part of the hydrogen peroxide vaporizer according to the present invention; and

FIG. 4 is a longitudinal sectional view showing the body part and a discharge part of the hydrogen peroxide vaporizer according to the present invention.

### MODE FOR INVENTION

Objects, characteristics and advantages of the present invention will be more clearly understood from the detailed description as will be described below and the attached drawings. Before the present invention is disclosed and described, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure.

Terms used in this application are used to only describe specific exemplary embodiments and are not intended to restrict the present invention. An expression referencing a singular value additionally refers to a corresponding expression of the plural number, unless explicitly limited otherwise by the context. In the description, when it is said that one portion is described as “comprises” and/or “comprising” any component, one element further may include other components unless no specific description is suggested. In the description, the same reference numerals will be used to describe the same components. It shall also be understood that the terms “and/or” used herein are intended to signify and include any or all possible combinations of one or more of the associated listed items, unless the context clearly indicates otherwise. It shall be understood that, although the terms “first,” “second,” etc. may be used herein to describe various components, the components should not be limited by these terms. These terms are only used to distinguish one component from another. For example, without departing from the scope of the present disclosure, a first component may be termed as a second component.

All terms used herein, including technical or scientific terms, unless otherwise defined, have the same meanings which are typically understood by those having ordinary skill in the art. The terms, such as ones defined in common dictionaries, should be interpreted as having the same meanings as terms in the context of pertinent technology, and should not be interpreted as having ideal or excessively formal meanings unless clearly defined in the specification.

Hereinafter, a desirable embodiment of the present invention will be explained in detail with respect to the attached drawings.

Configuration of Hydrogen Peroxide Vaporizer

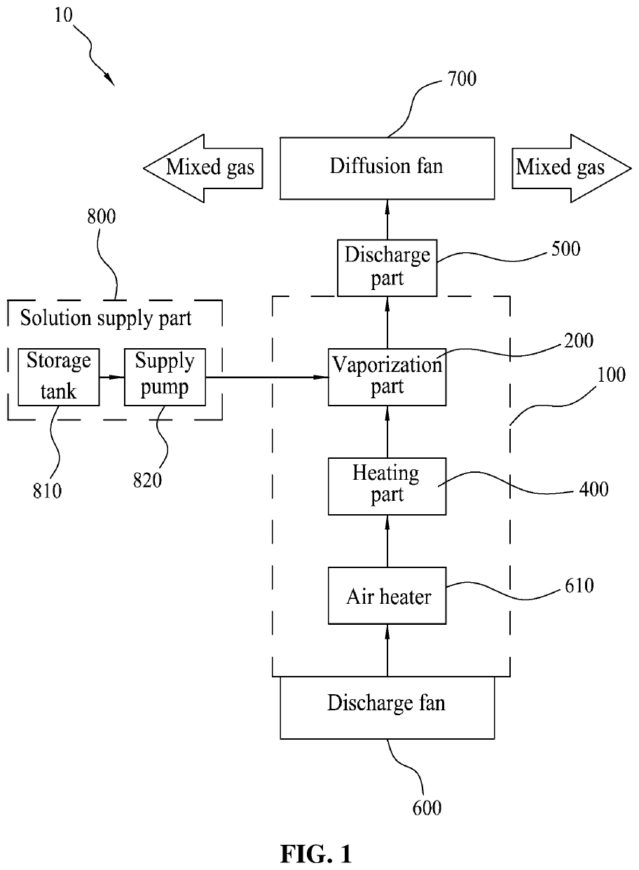

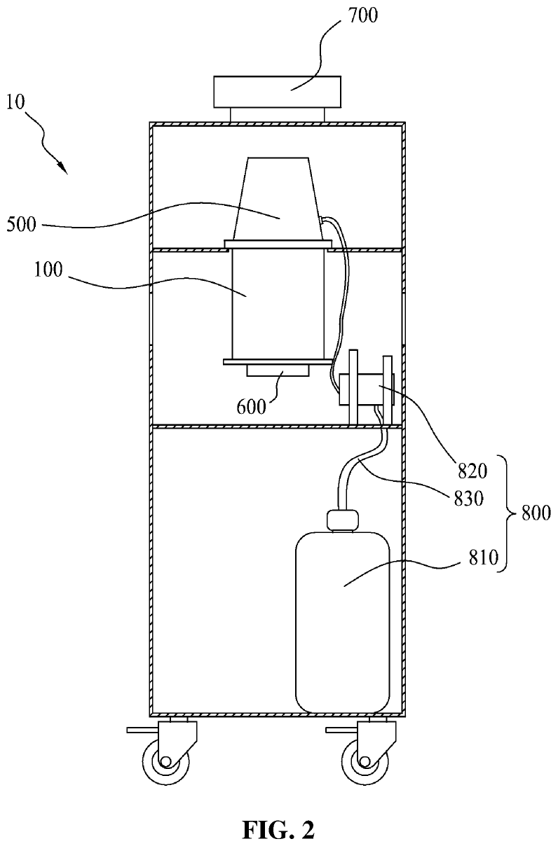

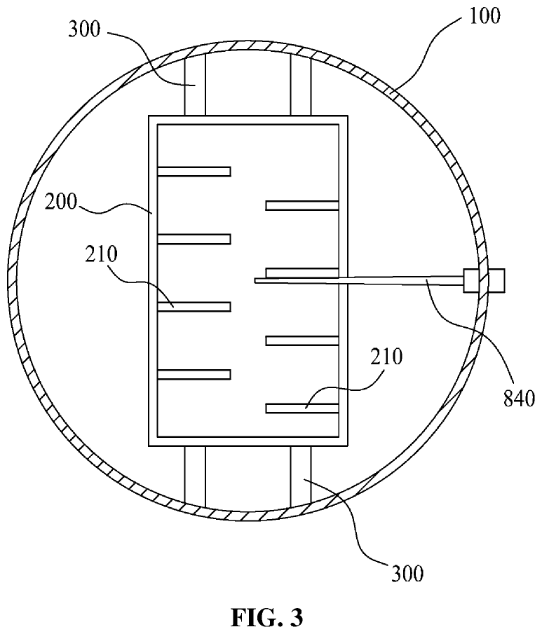

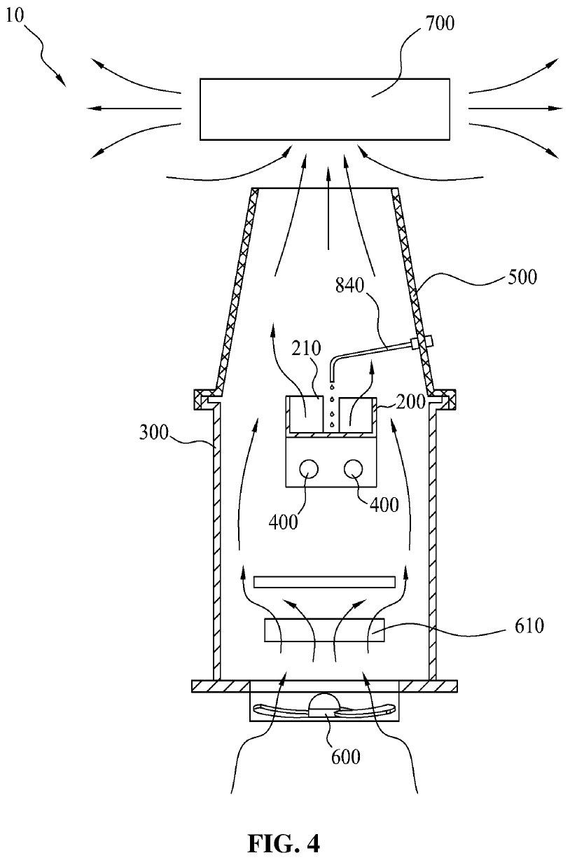

FIG. 1 is a block diagram showing a schematic configuration of a hydrogen peroxide vaporizer according to the present invention, FIG. 2 is a side view showing a sterilizer having the hydrogen peroxide vaporizer according to the present invention, FIG. 3 is a transverse sectional view showing a body part of the hydrogen peroxide vaporizer according to the present invention, and FIG. 4 is a longitudinal sectional view showing the body part and a discharge part of the hydrogen peroxide vaporizer according to the present invention. A hydrogen peroxide vaporizer 10 according to the present invention is applied to a sterilizer that performs sterilization with hydrogen peroxide vapor generated by vaporizing a hydrogen peroxide solution. As shown in FIGS. 1 to 4, the hydrogen peroxide vaporizer 10 largely includes a body part 100, a vaporization part 200, support parts 300, a heating part 400, a discharge part 500, a discharge fan 600, a solution supply part 800, and a diffusion fan 700.

The body part 100 provides an outer appearance of the hydrogen peroxide vaporizer 10 and spaces for seating parts constituting the hydrogen peroxide vaporizer 10. The body part 100 is made to various sizes in consideration of the capacity of the sterilizer in which the hydrogen peroxide vaporizer 10 is adopted, a sterilization area, and the like. Only if the body part 100 is hollow so that the parts of the hydrogen peroxide vaporizer 10 are coupled to the inside or outside thereof, the shape of the body part 100 does not matter. Desirably, the body part 100 has the shape of a hollow tube, column, or pipe whose top and underside are open and is circular in cross section. However, the body part 100 may be polygonal or irregular in cross section according to aspects of use thereof.

The vaporization part 200 is disposed on one side of the inside of the body part 100 and provides a place in which a hydrogen peroxide solution is accommodated and vaporized. The vaporization part 200 is disposed on one side of the inside of the body part 100, desirably, on one side of the lower portion of the inside of the body part 100, while being spaced apart from the inner walls of the body part 100 by a given distance by means of the support parts 300. In this case, only if the vaporization part 200 has wall surfaces with a given height extending from the edges thereof in such a way that a groove is formed in the center thereof to store the hydrogen peroxide solution supplied by given capacity, the shape of the vaporization part 200 does not matter. However, desirably, the vaporization part 200 has the shape of a hexahedron so that the heat transferred from the heating part 400 is uniformly transferred to the vaporization part 200. In this case, the vaporization part 200 has a given size so that about 1 to 22 g of a hydrogen peroxide solution, desirably, about 8 to 20 g of a hydrogen peroxide solution is vaporized per minute, and more desirably, the vaporization part 200 has a given size so that even if vaporization of the hydrogen peroxide solution is delayed for a predetermined time, the hydrogen peroxide solution does not overflow from the vaporization part 200. To do this, the vaporization part 200 has a width of 50 to 60 mm, a length of 80 to 100 mm, and a height of about 40 mm. If the vaporization part 200 has a height less than 30 mm, the hydrogen peroxide solution supplied from the solution supply part 800 may overflow from the vaporization part 200 or scatter to the outside of the vaporization part 200 due to fall repulsion. Contrarily, if the vaporization part 200 has a height greater than 50 mm, a temperature transferred from the heating part 400 is not transferred gently to the wall surfaces of the vaporization part 200, so that changes in temperatures of the wall surfaces of the vaporization part 200 may be seriously made to fail to achieve uniform vaporization of the hydrogen peroxide solution

Further, the vaporization part 200 is made of a high chemical resistant material, desirably an aluminum material so that it can be prevented from being corroded by the hydrogen peroxide solution. According to the present invention, for the convenience of the explanation, the vaporization part 200 has the shape of a hollow hexahedron whose top is open, but it may not be limited thereby. In this case, the vaporization part 200 has protrusions or partition walls 210 protruding from the inner bottom thereof or from at least one or more wall surfaces thereof to increase the contact area with the hydrogen peroxide solution accommodated therein so that a vaporization efficiency is improved. As the contact area of the vaporization part 200 with the hydrogen peroxide solution is increased, accordingly, the vaporization part 200 is decreased in volume, so that even while the entire volume of the hydrogen peroxide vaporizer 10 is being reduced, a high vaporization efficiency is obtained.

As mentioned above, the support parts 300 serve to stably support the vaporization part 200 disposed on one side of the inside of the body part 100. Only if the support parts 300 stably fix and support the vaporization part 200 disposed on the inside of the body part 100, the shapes of the support parts 300 do not matter. However, the support parts 300 desirably have minimum contact points through which a state in which the body part 100 and the vaporization part 200 are spaced apart from each other is kept, so that air generated from the discharge fan 600 as will be discussed later flows gently and minimizedly contacts with the body part 100 to minimize heat loss. For example, two or more bar-shaped support parts 300 are provided spaced apart from each other to form a passage through which air flows. If a single support part 300 stably supports the vaporization part 200, the single support part 300 is provided. However, it is not easy to acquire a sufficiently stable support force from the single support part 300, and accordingly, it is desirable that at least two or more bar-shaped support parts 300 be provided, and the distance between the support parts 300 may be set to various lengths in consideration of the capacity of the sterilizer, the vaporization efficiency of the vaporization part 200, and the heat loss rate of the vaporization part 200.

The heating part 400 is connected to one side of the vaporization part 200, generates heat for vaporizing the hydrogen peroxide solution accommodated in the vaporization part 200, and transfers the heat to the vaporization part 200. Only if the heating part 400 is a device that heats the vaporization part 200 to a temperature greater than a vaporized boiling point of the hydrogen peroxide solution, it does not matter. According to the present invention, heating rods are used as the heating part 400, and the heating rods are low-priced, simple in configuration, have high efficiency, and are easy to be controlled. In this case, two 1000 W heating rods are used to provide 2000 W heating part 400. Vaporization efficiencies are measured according to the capacities of the heating part 400, that is, 500 W, 1000 W, and 1500 W less than 2000 W, and as a result, it is found that the temperature of the vaporization part 200 is hard to be constantly kept. In specific, because of small capacity of the heating part 400, the changes in temperature of the vaporization part 200 happen seriously and drastically to fail to uniformly keep the temperature of the vaporization part 200, so that even the vaporization efficiency of the hydrogen peroxide solution is not constantly maintained.

It is desirable that the vaporization part 200 be kept to a temperature between about 130 to 150° C., preferably about 140° C. by means of the heating part 400. The comparison results between the changes in temperature of the vaporization part 200 and the vaporization efficiencies of the hydrogen peroxide solution are suggested in Table 1. In this case, the vaporization part 200 has a width of 54 mm, a length of 88 mm, and a height of 40 mm to store a maximum 24 ml hydrogen peroxide solution. While about 20 ml hydrogen peroxide solution is being supplied per minute to the vaporization part 200, an amount of vaporized hydrogen peroxide solution for one minute is measured.

TABLE 1

| | Amount of vaporized hydrogen |
|---|---|
| Temperature (° C.) | peroxide solution (g) |
| 120 | 10 |
| 130 | 14 |
| 140 | 16 |
| 150 | 14 |

As appreciated from Table 1, it is checked that in a process where the temperature of the vaporization part 200 increases to 140° C., the vaporization efficiency of the hydrogen peroxide solution is improved, but in a process where the temperature of the vaporization part 200 is greater than or equal to 150° C., the vaporization efficiency of the hydrogen peroxide solution is deteriorated. The vaporized boiling point of the hydrogen peroxide solution is about 140° C., and accordingly, at the temperature of 150° C., the hydrogen peroxide solution supplied to the vaporization part 200 starts to be vaporized and then exploded to the outside of the vaporization part 200 to cause an accident risk, so that the hydrogen peroxide solution is not vaporized well and scatters to the outside of the vaporization part 200, thereby making the vaporization efficiency deteriorated. Contrarily, if the temperature of the vaporization part 200 is less than or equal to 120° C., the vaporization efficiency of the hydrogen peroxide solution becomes deteriorated and the amount of hydrogen peroxide solution supplied is larger than the amount of vaporized hydrogen peroxide solution, so that as time passes, the hydrogen peroxide solution may overflow from the vaporization part 200. Therefore, it can be appreciated that when the vaporization part 200 is kept to a temperature between about 130 to 150° C., preferably about 140° C. by means of the heating part 400, a maximum vaporization efficiency is obtained stably. To do this, the heating part 400 has two heating rods each having a capacity of about 1000 W. The discharge part 500 is provided on the open top of the body part 100, prevents the diffusion efficiency of the hydrogen peroxide vapor generated from the vaporization part 200 from being deteriorated due to arbitrary diffusion of the hydrogen peroxide vapor, and thus induces the hydrogen peroxide vapor to be discharged gently to the diffusion fan 700 for diffusing the hydrogen peroxide vapor. The discharge part 500 is tapered upward so that the hydrogen peroxide vapor generated from the vaporization part 200 moves up and is thus collected to the center thereof, and through an air jet effect, the discharge speed of the hydrogen peroxide vapor is increased. That is, a diameter of the underside of the discharge part 500 is equal to a diameter of top of the body part 100, and the diameter of the discharge part 500 becomes gradually reduced toward top of the discharge part 500. In this case, desirably the diameter of top of the discharge part 500 is 30 to 50% of an inlet diameter of the diffusion fan 700 so that the vapor discharged from the discharge part 500 is introduced into the diffusion fan 700, without any loss. Further, the discharge part 500 has a given length so that the hydrogen peroxide vapor generated from the vaporization part 200 is quickly discharged to the outside before it turns into water drops, and the length of the discharge part 500 may be freely adjusted according to the capacity of the vaporization part 200. Desirably, a distance from the vapor generation surface of the vaporization part 200 to the top of the discharge part 500 is between about 200 and 300 mm, preferably about 250 mm. Further, the discharge part 500 desirably has the sectional shape of a cylinder so that the high temperature vapor can be prevented from flowing back to the bottom due to convection or the resistance of the vapor to air flow like whirlwind can be minimized, thereby being quickly introduced into the diffusion fan 700. If the sectional shape of the discharge part 500 is circular, the discharge part 500 achieves more efficient temperature control than a discharge part having the sectional shape of a polygon. The discharge fan 600 is disposed on one side of the inside of the body part 100 and supplies air with which the hydrogen peroxide vapor is mixed to the discharge part 500 so that the hydrogen peroxide vapor generated from the vaporization part 200 moves to a target in the state of vapor, without being cold and thus liquefied again, while moving. Only if the discharge fan 600 discharges the air with which the hydrogen peroxide vapor is mixed gently, the installation position of the discharge fan 600 does not matter, but desirably, the discharge fan 600 is disposed on one side of the lower portion of the inside of the body part 100. More desirably, the discharge fan 600 is disposed at a relatively lower position than the vaporization part 200 and the heating part 400 inside the body part 100, so that the air supplied from the discharge part 600 passes through the heating part 400, is heated to a given temperature, and is mixed with the hydrogen peroxide vapor.

Further, an air heater 610 is disposed on one side of the inside of the body part 100, desirably between the discharge fan 600 and the vaporization part 200, to heat the air introduced through the discharge fan 600. The air heater 610 heats the air introduced through the discharge fan 600 to a given temperature so that the air of the interior of the body part 100 and the hydrogen peroxide vapor move upward easily, and since the hydrogen peroxide vapor is sensitive to a temperature, further, the air heater 610 serves to mix the air supplied from the discharge fan 600 with the hydrogen peroxide vapor, in a state where the air is heated, so that the hydrogen peroxide vapor can be completely diffused, without being condensed. Only if the air heater 610 heats the air to a given temperature, preferably to a temperature between 50 to 70° C., more preferably to about 60° C., the type of the air heater 610 does not matter.

The diffusion fan 700 is disposed spaced apart from top of the discharge part 500 by a given distance and serves to diffuse the hydrogen peroxide vapor discharged from the discharge part 500. Only if the diffusion fan 700 gently diffuses the hydrogen peroxide vapor discharged from the discharge part 500, the type of the diffusion fan 700 does not matter. In this case, a distance between the introduction portion of the diffusion fan 700 and the discharge part 500 is freely determined according to the use capacity. If the distance between the introduction portion of the diffusion fan 700 and the discharge part 500 is long, a large amount of surrounding air is mixed with the hydrogen peroxide vapor so that before the hydrogen peroxide vapor is introduced into the diffusion fan 700, it may be condensed or some of the hydrogen peroxide vapor are discharged to the outside of the diffusion fan 700 to make the diffusion efficiency deteriorated. Contrarily, if the distance between the introduction portion of the diffusion fan 700 and the discharge part 500 is short, a suction force of the diffusion fan 700 is increased so that before the air supplied from the discharge fan 600 is sufficiently heated, it is introduced into the diffusion fan 700 or not gently mixed with the hydrogen peroxide vapor, and the hydrogen peroxide solution scattering from the vaporization part 200 is introduced into the diffusion fan 700, in a state of being not vaporized. Accordingly, the distance between the diffusion fan 700 and the discharge part 500 should be determined in consideration of the use capacity and surrounding environments.

The capacity of the discharge fan 600 is desirably determined according to the capacity of the diffusion fan 700. According to the present invention, the discharge fan 600 has the capacity of 100 to 150 m³/h, and the diffusion fan 700 has the capacity of 500 to 1500 m³/h. Preferably, the discharge fan 600 has the capacity of about 120 m³/h, and the diffusion fan 700 has the capacity of about 1050 m³/h. Desirably, the top area of the discharge part 500 is smaller than the introduction portion area of the diffusion fan 700 into which air is introduced. For example, if the introduction portion area of the diffusion fan 700 is 155 Φ, the top area of the discharge part 500 is 100 Φ. The top area of the discharge part 500 is desirably about 40% of the introduction portion area of the diffusion fan 700 so that the hydrogen peroxide vapor discharged from the discharge part 500 is perfectly introduced into the diffusion fan 700, without leaking to the outside of the diffusion fan 700.

The solution supply part 800 is disposed on one side of the body part 100 or spaced apart from the body part 100 by a given distance and serves to supply the hydrogen peroxide solution to the vaporization part 200. The solution supply part 800 includes a storage tank 810 in which the hydrogen peroxide solution is stored, a supply hose 830 connected to the storage tank 810, a supply nozzle 840 disposed at one end of the supply hose 830, and a supply pump 820 for applying a pressure for the supply of the hydrogen peroxide solution. The solution supply part 800 has the same or similar configuration as or to general supply devices for supplying liquids, and accordingly, a detailed explanation of the configuration of the solution supply part 800 will be avoided. In this case, the end portion of the supply nozzle 840 of the solution supply part 800 is spaced apart from the bottom of the vaporization part 200 by a distance of about 10 to 20 min, preferably about 15 mm. Further, the solution supply part 800 supplies about 10 to 30 ml of the hydrogen peroxide solution per minute, preferably, about 20 ml of the hydrogen peroxide solution per minute.

Use Situations of Hydrogen Peroxide Vaporizer

Under the above-mentioned configuration, an explanation of the use situations of the hydrogen peroxide vaporizer 10 according to the present invention will be given in detail below.

First, the body part 100 is located on a place where sterilization is needed or on a sterilizer.

Next, the vaporization part 200 is heated to a temperature at which the hydrogen peroxide solution is vaporized using the heating part 400. In this case, as shown in FIG. 4, the heating part 400 consists of two heating rods parallel with each other to transfer uniform heat to the entire vaporization part 200. Further, the vaporization part 200 is fixed to the body part 100 with minimum contact points with the body part 100 through the support parts 300, thereby enabling the heat loss caused by the body part 100 to be minimized. Accordingly, the heat loss generated when the heat supplied from the heating part 400 comes into contact with external air is minimized to thus optimize the heating efficiency. Further, the vaporization part 200 is disposed on the center of the body part 100, while being spaced apart from the body part 100 through the support parts 300, so that even if a fire happens in the vaporization part 200, the vaporization part 200, which is distant from surrounding combustible materials, ensures the safety of use.

Next, the hydrogen peroxide solution is supplied from the solution supply part 800 to the vaporization part 200. The hydrogen peroxide solution is first supplied to the supply hose 830 connected to the storage tank 810, and next, the supply nozzle 840 disposed at the end of the supply hose 830 supplies the hydrogen peroxide solution to the vaporization part 100. An amount of hydrogen peroxide solution supplied from the solution supply part 800 is controlled by the adjustment of the pressure of the supply pump 820 in consideration of the capacity and vaporization speed of the vaporization part 200.

After that, the hydrogen peroxide solution supplied to the vaporization part 200 is vaporized in the vaporization part 200 heated through the heating part 400 and produced as hydrogen peroxide vapor. In this case, even if the hydrogen peroxide solution supplied to the vaporization part 200 scatters from the bottom of the vaporization part 200 because of its drop, the scattering hydrogen peroxide solution comes into contact with the wall surfaces of the vaporization part 200 and is thus vaporized, thereby improving the vaporization efficiency. Further, if the vaporization part 200 has the protrusions or partition walls 210, the contact area of the hydrogen peroxide solution with the protrusions or partition walls 210 is increased to optimize the vaporization efficiency of the hydrogen peroxide solution. As a result, the area of the vaporization part 200 can be reduced, thereby enabling the whole volumes of the hydrogen peroxide vaporizer 10 and the sterilizer to be decreased. In this case, desirably, the vaporization part 200 has a given size so that about 1 to 22 g of a hydrogen peroxide solution, preferably about 8 to 20 g of a hydrogen peroxide solution is vaporized per minute.

Next, the hydrogen peroxide vapor generated from the vaporization part 200 is mixed with air by means of the discharge fan 600 and thus discharged through the discharge part 500. In this case, the discharge part 500 is tapered upward so that the hydrogen peroxide vapor generated from the vaporization part 200 is gently mixed with the air supplied from the discharge fan 600, without arbitrarily scattering, and thus discharged to a discharge hole designated by a user. Further, the air introduced through the discharge fan 600 passes through the air heater 610, is heated to a temperature of about 60° C., and primarily mixed with the hydrogen peroxide vapor generated from the vaporization part 200. Like this, before the hydrogen peroxide vapor is discharged through the discharge part 500, the hydrogen peroxide vapor is discharged in the state of being primarily mixed with the high temperature air, so that the temperature of the hydrogen peroxide vapor is not easily reduced, while being diffused. As a result, the hydrogen peroxide vapor is gently diffused, without being condensed. Moreover, the primary mixture stably moves upward from the discharge part 500, without any backflow, by means of the pressure of the discharge fan 600 and the heated air.

Lastly, the primary mixture between the hydrogen peroxide vapor and the air discharged through the discharge part 500 is secondarily mixed with the surrounding air before introduced into the diffusion fan 700 and then introduced into the diffusion fan 700. Like this, through the secondary mixing between the hydrogen peroxide vapor and the air, the concentration of hydrogen peroxide contained in the air becomes lowered, thereby enabling stable sterilization. The secondary mixture is diffused through the diffusion fan 700 and thus sterilizes the space, place, or article designated by the user. In this case, the discharge fan 600 and the diffusion fan 700 are provided to have optimized diffusion capacities in consideration of a sterilization range and a vapor supply amount.

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above teachings. It is therefore intended that the scope of the invention be limited not by this detailed description, but rather by the claims appended hereto and the equivalents thereof.

1. A hydrogen peroxide vaporizer for use in a sterilizer for performing sterilization with hydrogen peroxide vapor generated by means of the vaporization of a hydrogen peroxide solution, the hydrogen peroxide vaporizer comprising:
a body part having the shape of a hollow column;
a vaporization part disposed on one side of the inside of the body part and having a predetermined capacity to accommodate the hydrogen peroxide solution;
support parts for supporting the vaporization part to allow the vaporization part to be spaced apart from the inside of the body part by a predetermined distance;
a heating part disposed on one side of the vaporization part to heat the vaporization part; and
a discharge part disposed on top of the body part to induce the hydrogen peroxide vapor generated from the vaporization part to be discharged.

2. The hydrogen peroxide vaporizer according to claim 1, wherein the vaporization part has protrusions or partition walls protruding from the inner bottom thereof or at least one or more wall surfaces thereof to increase the contact area with the hydrogen peroxide solution.

3. The hydrogen peroxide vaporizer according to claim 1, wherein the discharge part becomes gradually narrow to the top from the bottom thereof.

4. The hydrogen peroxide vaporizer according to claim 1, further comprising a discharge fan disposed on one side of the inside of the body part to easily discharge the hydrogen peroxide vapor generated from the vaporization part to the discharge part.

5. The hydrogen peroxide vaporizer according to claim 1, wherein the discharge fan is disposed at a relatively lower position than the vaporization part.

6. The hydrogen peroxide vaporizer according to claim 1, further comprising a solution supply part disposed on one side of the body part to supply the hydrogen peroxide solution to the vaporization part.

7. The hydrogen peroxide vaporizer according to claim 1, further comprising a diffusion fan disposed on top of the discharge part or spaced apart from top of the discharge part by a predetermined distance.
